(19) United States
(12) Patent Application Publication
(10) Pub. No.: US 2026/0240180 A1
(43) Pub. Date: Aug. 20, 2026

(54) NANOBUBBLES FOR TREATING PERISHABLE PRODUCTS

(71) Applicant: Messer Canada Inc., Mississauga (CA)

(72) Inventors: Donna Lynn Leger, Burlington (CA); Calvin Knaggs, Brampton (CA)

(73) Assignee: Messer Canada Inc., Mississauga, ON (CA)

(21) Appl. No.: 19/489,627

(22) PCT Filed: Jun. 20, 2024
(86) PCT No.: PCT/CA2024/050831

§ 371 Date: Dec. 3, 2025

Related U.S. Application Data
(60) Provisional application No. 63/522,440, filed on Jun. 22, 2023.

(51) Int. Cl. A23B 11/10; A23C 9/152

(52) U.S. Cl. CPC A23B 11/10; A23C 9/1524

(57) ABSTRACT

A fluid composition having reduced susceptibility to at least one of oxidative spoilage and of microbial spoilage is provided, wherein the fluid composition consists of nanobubbles of gas, and each nanobubble in the fluid composition ranges in size from 1 nanometer to 500 nanometers to reduce the at least one of the oxidative spoilage and of the microbial spoilage. There is also provided a process for reducing a fluid composition susceptibility to at least one of oxidative spoilage and of microbial spoilage which consists of mixing nanobubbles of gas into the fluid composition, and each nanobubble in the fluid composition ranges in size from 1 nanometer to 500 nanometers for reducing the at least one of the oxidative spoilage and of the microbial spoilage.

### BACKGROUND OF THE INVENTION

The present embodiments relate to the use of gases, and compositions which include gases, including but not limited to oxygen, to treat perishable fluids such as for example perishable liquid food products.

Perishable fluids, which include perishable liquids, may be edible liquids for example that are likely to spoil, decay or become unsafe to use or consume if not kept refrigerated at a temperature of 40° F. (4.4° C.) or below, or frozen at 0° F. (−17.8° C.). An example of one type of perishable liquid would be dairy products which have a “best before” date, wherein the consumer is notified of a date after which the liquid product may be undesirable. For purposes herein, a “perishable fluid” and a “perishable liquid” may be used interchangeably unless specified otherwise.

These perishable liquids are susceptible to spoilage and have a limited shelf life due to microbial. growth or oxidation in the liquid over time; even if the liquid has undergone treatment, such as pasteurization, to extend shelf life and best before date.

For example, milk will typically have a post-pasteurization shelf life of 12-14 days, depending upon the fat level and flavorings mixed into the milk. The integrity of the cold supply chain to transport the milk post-pasteurization will also impact the shelf life of the milk.

Milk spoilage or reduced shelf life of the milk may also occur as a result of bacteria in the milk, possible coming from the container holding the milk. These effects will typically be seen in the organoleptic properties of the milk which may be compromised in the texture, flavor, and overall quality of the milk. There are a variety of known acid-producing bacteria in milk such as: Enterococcus, Lactobacillus, Lactococcus, Leuconostoc, Pediococcus, and Streptococcus, each one of which can create the well-known and typical “sour” taste associated with spoiled milk.

In general, exposure to oxygen (O₂) is considered detrimental to the quality and shelf life of milk and other dairy products. For example, when milk is exposed to oxygen in the air, the lactoglobulin in the milk can bind with the oxygen which causes the milk to develop off-flavors and a slightly yellowish tint or color.

Similarly, excessive or high oxygen concentration in yogurt (a semi-solid food fluid) can have a negative impact on the quality of the yogurt and promote the growth of unwanted bacteria in the yogurt which will spoil same, thereby negatively affecting the yogurt taste, texture, and nutritional properties.

It is common practice in the food and beverage industry to add gas or gas compositions to liquids for improving the texture or organoleptic quality of the liquids. Carbon dioxide (CO₂) is frequently used in carbonated beverages such as, for example, soda or sparkling water, while nitrogen (N₂) is added to some premium beers such as Guinness® beer.

The size of the bubbles used in liquid products, such as perishable liquid products, differ depending upon the type of liquid to be used or consumed. For example and by reference to a beer, carbon dioxide used in low carbonated waters will typically have an average bubble diameter of approximately (~) 0.5 mm. Beer, however, will include therein bubbles that each average closer to 1 millimeter (mm). In contrast, the nitrogen bubbles in a Guinness beer are only about a tenth of a millimeter.

The food and beverage industry, in particular, continues to seek a method to provide a more reliable process to increase the shelf life of perishable liquid products, especially liquid food products such as, for example, milk.

Limited shelf life has costs for the food industry. Every additional day of shelf life is an economic benefit for producers and retailers of perishable liquids. As mentioned above, companies use pasteurization and microfiltration methods to extend shelf life in perishable liquids. These known methods will lower the overall microbial count, but at a significant cost to the producer. Pasteurization, in particular, can impact the taste and texture (the organoleptic qualities) of the final liquid product.

Moreover, it is counterintuitive to use oxygen as a method to extend shelf life. The food and beverage industry considers the addition of oxygen in food products, liquid or otherwise, to cause negative organoleptic characteristics and lead to a product that will spoil much more quickly than would otherwise occur. That is why it is common for producers and processors to inert or limit any oxygen from the presence of the food products.

### SUMMARY OF THE INVENTION

According to an illustrative embodiment of the present invention, provided is a fluid composition having reduced susceptibility to at least one of oxidative spoilage and of microbial spoilage, wherein the fluid composition consists of nanobubbles of gas, and each nanobubble in the fluid composition ranges in size from 1 nanometer to 500 nanometers to reduce the at least one of the oxidative spoilage and of the microbial spoilage.

According to another illustrative embodiment of the present invention there is provided a process for reducing a fluid composition susceptibility to at least one of oxidative spoilage and of microbial spoilage which consists of mixing nanobubbles of gas into the fluid composition, and each nanobubble in the fluid composition ranges in size from 1 nanometer to 500 nanometers for reducing the at least one of the oxidative spoilage and of the microbial spoilage.

According to other illustrative embodiments of the present invention, provided are a fluid composition and process, both with nanobubbles of gas, for a food product selected from the group consisting of milk, yogurt, a dairy product other than milk and yogurt, beer, fruit juice, vegetable juice, vegetable oil, vegetable puree, fruit puree, flavored water, and wine; and for a product consisting of latex paint, a cosmetic, liquid soap, and a pharmaceutical.

According to still another illustrative embodiment of the present invention, the nanobubbles of gas are selected form the group consisting of, but not limited to, oxygen (O₂) nanobubbles, carbon dioxide (CO₂) nanobubbles, nitrogen (N₂) nanobubbles, ozone (O₃) nanobubbles, nitrous oxide (N₂O) nanobubbles, and mixtures thereof.

Nanobubbles, each of which measure less than 200 nanometers (nm) in diameter, are one of the smallest known bubble sizes. Nanobubbles can remain suspended in liquid for many weeks without rising to the surface and off-gassing to atmosphere, unlike known larger bubble size gases used in carbonated beverages. This is because nanobubbles are significantly smaller by several orders of magnitude than either CO₂ gas or N₂ gas used in known beverages. Nanobubbles with a diameter each in the range of 70-120 nm are 1000 times smaller than each of the bubbles used in known beverage gases.

Because nanobubbles have a smaller ratio of surface area to volume (surface area: volume) in solution, and because nanobubbles remain suspended in liquid for a longer duration of time than larger bubbles, it is possible to provide a greater concentration of gas into a liquid using nanobubbles than can be done with other gas methods.

Nanobubbles exhibit different physical and chemical properties compared to larger scale bubbles of the same gas in solution or dissolved gases. Nanobubbles may exhibit different physical and chemical properties at the gas/liquid interface in the liquid, and each nanobubble or a myriad of nanobubbles may exhibit a small electrical charge at their surface or surfaces. Nanobubbles in solution over a period of time may reduce the level of the same gas dissolved in the solution by scavenging the dissolved gas, as compared to a liquid not treated with nanobubbles. In effect, the nanobubbles scavenge the free dissolved gas in the solution and thereby, bring the dissolved gas into the nanobubbles.

The benefits of the claimed embodiments using the nanobubble treatments include, but are not limited to: changing the viscosity of the perishable liquid, extending shelf life of a perishable liquid product by interacting-likely by clustering at the surface-with the microbial flora in the product, changing behavior of the microbes due to the clustering at or proximate to the surface of the microbes in the liquid, and/or reducing oxidation of the liquid by scavenging dissolved O₂ or other gases in the perishable liquid. By “scavenging” it is meant that gas nanobubbles may act via physical or chemical characteristics to remove unwanted gases from a fluid.

### BRIEF DESCRIPTION OF THE DRAWINGS

For a more complete understanding of the present embodiments, reference may be had to the following description of exemplary embodiments considered in connection with the accompanying drawing Figures, of which:

FIG. 1 shows an apparatus for providing a composition and a process for treating perishable products with nanobubbles according to an embodiment of the present invention.

FIG. 2 shows a flow chart of the process for treating perishable products with nanobubbles according to the present embodiments.

FIG. 3 shows a graph of the results of the introduction of nanobubbles of oxygen into a perishable liquid—milk—and the impact on shelf life of the milk as measured by odor of same, according to the present embodiments.

### DETAILED DESCRIPTION OF THE INVENTION

Before explaining the inventive embodiments in detail, it is to be understood that the invention is not limited in its application to the details of construction and arrangement of parts illustrated in the accompanying drawings, if any, since the invention is capable of other embodiments and being practiced or carried out in various ways. Also, it is to be understood that the phraseology or terminology employed herein is for the purpose of description and not of limitation.

In the following description, terms such as a horizontal, upright, vertical, above, below, beneath and the like, are to be used solely for the purpose of clarity illustrating the invention and should not be taken as words of limitation. The drawings are for the purpose of illustrating the invention and are not intended to be to scale.

The amount of dissolved oxygen in a perishable liquid may be reduced upon exposure of the perishable liquid to nanobubbles of oxygen. Gas scavenging in liquids herein refers to the process of reducing if not removing the presence of dissolved gases in a liquid medium, and such scavenging can be used to minimize if not eliminate the negative effects of oxidation.

Treating liquids with nanobubbles of oxygen, each nanobubble of which ranges in size from a few nanometers to a few hundred nanometers in diameter, provides oxygen bubbles with unique properties. For example, oxygen nanobubbles, due to their small size and large surface area, can significantly increase the interfacial area between the gas phase and liquid phase. This increased surface area facilitates enhanced mass transfer phenomena, such as improved gas dissolution or extraction of dissolved substances from the liquid.

Oxygen nanobubbles having a high affinity for the dissolved oxygen in solution will promote the dissolved oxygen's removal from the liquid phase and incorporation into the nanobubble itself. And, once the oxygen nanobubbles have interacted with the dissolved oxygen in solution, the resulting product should be less soluble, thereby allowing for the separation of gas-depleted liquid from the scavenging oxygen. In our Example (test) further described below, the oxygen dissolved in milk and treated with oxygen nanobubbles resulted in a drop of the oxygen level to a level lower than that of the oxygen level in the control group, and resulted in and extension of the shelf life.

The term “admixing” and any variants as used herein refer to and include “to mix in”, “to provide to”, to inject into“, ”to introduce into, and “to deliver to”. It is to be understood that as used and described herein the fluid composition embodiments and the process embodiments can reduce susceptibility to either of oxidative spoilage or microbial spoilage, or to both oxidative spoilage and microbial spoilage, i.e., to at least one of the oxidative spoilage and microbial spoilage.

The present embodiments call for the use of nanobubbles of gas, particularly but not limited to oxygen, to treat a perishable liquid product such as a food product to extend the shelf life and properties of the perishable liquid. The present embodiments call for treating a perishable substance, such as for example, a fluid composition susceptible to at least one of oxidative spoilage and microbial spoilage, which may include a perishable semi-solid product (such as yogurt, for example), a perishable fluid and a perishable liquid.

Other products to be treated with the nanobubbles include for example paint, cosmetics, soap (latex or otherwise) and pharmaceuticals.

Another exemplary embodiment calls for an optional degassing step used either prior to the nanobubble treatment and/or after the nanobubble treatment to extend the shelf life and properties of the perishable liquid. In milk, one of the main acid-producing spoilage bacteria is Lactobacillus.The typical size of a single bacterium is 0.6-0.9 μm in width and 1.5-6.0 μm in length, i.e., 100 times larger than a typical nanobubble.

Since nanobubbles are significantly smaller than the Lactobacillus bacterium, the surface of an oxygen nanobubble has a charge that will attract it to the surface of the bacteria, thereby attracting the nanobubbles of oxygen to cluster at the surface of the bacterium and inhibit its ability to grow. Given the relative size of the Lactobacillus bacterium versus that of a nanobubble, it is possible that greater than (>) 100 nanobubbles could surround a single bacterium, thereby preventing the bacterium from growing. Lactobacillus is considered “oxygen-tolerant” and therefore, this bacteria will not thrive (i.e., not grow or flourish) in an oxygen-rich environment Because of this, a Lactobacillus bacterium that is surrounded by oxygen nanobubbles will likely go into a state of stasis, whereby its metabolism will be reduced or slow down, thereby delaying the spoilage process. This is known as a bacteriostatic effect.

Certain gas nanobubbles may be able to penetrate a cell wall of a bacterium and kill the bacterium, in which case the nanobubbles would have a bactericidal effect, not just a bacteriostatic effect. The bactericidal effect may be further enhanced by the addition of a gas, such as ozone (O₃) for example, which gas has antimicrobial properties due to a destructive oxidative effect on cell walls. In a complex matrix such as a dairy beverage, a similar effect may occur where the charge and size of the nanobubbles will cause same to cluster around some of the bacteria or proteins in the matrix. Both effects of nanobubbles, i.e., the nanobubbles clustering around either bacteria or proteins, will cause a drop in the oxygen available for detection by a standard oxygen sensor positioned in the liquid.

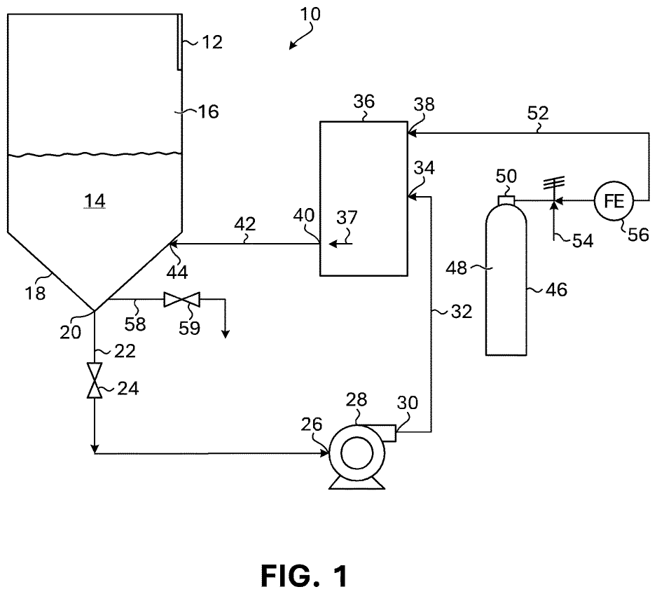

FIG. 1 shows an apparatus 10 embodiment of the present invention for admixing nanobubbles, such as for example oxygen nanobubbles, into a perishable fluid such as a perishable liquid or a perishable product or a perishable substance, such as for example milk. The apparatus includes a storage tank 12 or vessel in which is contained a perishable product 14, such as for example a perishable liquid or a perishable semi-solid product. The perishable product 14 may be introduced into the storage tank 14 by any of the known mechanisms or procedures to do so. Venting or exhausting of an interior space 16 of the storage tank 12 can be by any of the known mechanisms or procedures to do so.

A bottom 18 of the storage tank 12 is provided with an outlet 20 to which an end of a pipe 22 or conduit is connected for fluid communication with the perishable product 14 in the storage tank. A valve 24 is interposed in the pipe 22 for controlling fluid flow through the pipe. An opposite end of the pipe 22 is connected to an inlet 26 of a pump 28 for drawing the perishable product 14 from the storage tank 12 through the pipe to the pump. Alternatively, the perishable product 14 can be discharged from the bottom 18 of the storage tank 12 to the pump 28 under the effect of gravity. The pump 28 is provided with an outlet 30.

The outlet 30 of the pump 28 is in fluid connection with an end of another pipe 32 or conduit, while an opposite end of the pipe 32 is connected to a first inlet 34 of a nanobubble generator 36. The nanobubble generator 36 includes a second inlet 38 for a purpose to be described below, and an outlet 40 in fluid connection with an end of a pipe 42 or conduit. An opposite end of the pipe 42 is in fluid connection with an inlet 44 to the storage tank 12.

A gas cylinder 46 or vessel contains a gas 48 therein, and such gas is used in the production of the nanobubbles in the nanobubble generator 36. In that regard, the gas cylinder 46 includes a valve 50 through which the gas 48 can be released from the cylinder into a pipe 52 in fluid connection with the valve. A regulator 54 and a flow meter 56 are interposed in the pipe 52. An end of the pipe 52 opposite to the end connected with the valve 50 is in fluid connection with the second inlet 38 of the nanobubble generator 36.

In operation, the pump 28 (if not gravity) transfers the perishable product 14 from the storage tank 12 through the pipe 22 at a desired flow rate and discharge pressure into the pipe 32 to the nanobubble generator 36. The gas 48 from the gas cylinder 46 is metered and pressurized for transit through the pipe 52 and the inlet 38 into the nanobubble generator 36, so that an optimum volume of gas 48 becomes nanobubbles in the generator 36 for being admixed with the perishable product 14. A concentration of the nanobubbles 37 provided from the generator 36 into the perishable product 14 is determined by how long the perishable product 14 in the storage tank 12 is recirculated through the apparatus 10.

When a desired concentration of gas nanobubbles has been achieved and/or the gas sensor in the treated perishable product 14 reaches the desired level, the perishable product can be removed from the storage tank 12 via a discharge pipe 58 for storage, further processing or packaging. The discharge pipe 58 includes a valve 59 interposed therein.

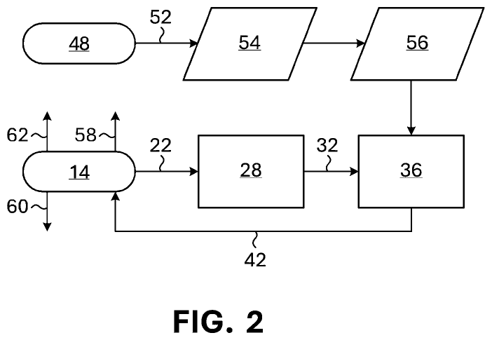

Referring now to FIG. 2 there is shown a flow chart of the process embodiment, wherein the steps of the process correspond to that which is shown in FIG. 1, and further that the disposition of other gas or gases in the perishable fluid or perishable product is set forth. Additional embodiments of the present invention include a preliminary step of degassing the perishable product or liquid to be treated of undesirable gases prior to providing the nanobubbles into the perishable product or liquid, and optionally or in addition thereto, a post-treatment degassing step to remove any larger than necessary or undesired gas bubbles from the nanobubble-treated perishable product or liquid. That is, any gas or gases other than the nanobubbles may be (i) removed 60 from the perishable fluid 14 before the admixing of the nanobubbles 37 in the perishable fluid, or (ii) removed 62 after the admixing of the nanobubbles 37 in the perishable fluid, or (iii) removed before 60 and removed 62 after the admixing of the nanobubbles 37 in the fluid.

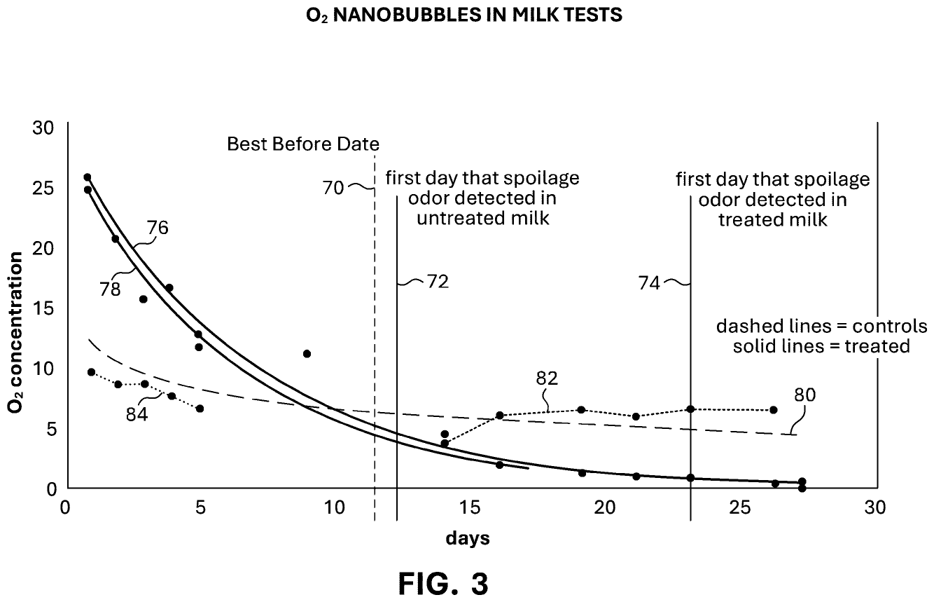

FIG. 3 shows the results of the oxygen level testing in milk in both an untreated control(s), and an oxygen nanobubble treated sample(s). The treated samples started with oxygen levels significantly higher than the untreated control samples. The vertical bars show the “Best Before Date”, the first day when a sniff test reported a sour smell for the untreated control sample, and the first day when a sniff test reported a sour smell for the nanobubble-treated sample.

Examples. The experiments treated samples of milk from the same milk portion with nanobubbles of O₂, and N₂O. The samples were tested for oxygen concentration and pH, as well as odor, and samples were opened throughout a twenty-eight (28) day period for testing. The Best Before Date for the samples was day 13. Samples were refrigerated during the test period.

### EXAMPLE 1

### Oxygen (O₂) Nanobubbles in a Perishable Liquid—Milk

FIG. 3 represents the results of treating a sample of milk with oxygen nanobubbles and measuring the oxygen concentration over time using an oxygen sensor. The samples were tested over a twenty-seven (27) day period, wherein separate samples that had been stored in a refrigerator were opened and tested for both oxygen levels and a “sniff” (olfactory) test by at least two individuals to detect possible spoilage.

The results of the testing are shown in FIG. 3. The x-axis of the graph is the number of “days”. The dotted vertical line 70 at day twelve (12) represents the manufacturer's Best Before Date. The solid vertical line 72 at day thirteen (13) represents the first date where at least one of the sniff testers first detected a sour smell (first day that spoilage odor detected in untreated milk), thereby indicating spoilage in the control samples. Sniff testers continued to detect sour smells in the subsequent days following day thirteen (13).

The solid vertical line 74 at day twenty-three (23) represents the first date where at least one of the sniff testers first detected a sour smell indicating spoilage in the nanobubble-treated samples (first day that spoilage odor detected in treated milk). The first indication of spoilage in the treated samples was, therefore, ten (10) days later than in the control samples.

The solid curved lines 76,78 in FIG. 3 represent the results of measurements taken over time by the oxygen sensor of the samples. The y-axis is the mg/L (milligrams per liter) of oxygen that is detected by the sensor (the O₂ concentration). The dotted or broken lines 80,82,84 all represent “control” samples with no treatment. The initial reading of the untreated milk was 13 mg/L of oxygen. Over twenty-seven (27) days, the control sample oxygen 80 reading dropped slowly to about 5 mg/L. The best fit curve broken line 80 describes the change in oxygen levels.

The solid lines 76,78 (treated) represent results for the oxygen nanobubble-treated samples. The initial treatment 76 led to a level of oxygen in excess of 25 mg/L—significantly higher than the control sample. The curves are computer-generated “best fits” for the measured oxygen levels for the remaining twenty-seven (27) days.

As shown in FIG. 3, the oxygen levels dropped to the point where on or about day ten (10), the measured oxygen in the control 80,82,84 and the nanobubble-treated samples 76,78 were approximately equivalent. In days subsequent to day ten (10), the oxygen levels continued to drop lower in the samples (76,78) than that of the control. These were unexpected results.

Since the samples were in a sealed and controlled environment, it is unlikely that the oxygen was entirely due to off-gassing and even if there was a small amount of off-gassing, it is unlikely that there would be more off-gassing from the treated sample versus the control.

It is more likely that the oxygen nanobubbles remained in the nanobubble-treated milk matrix. Given that there was a measurable impact on spoilage in the testing, and that nanobubbles of oxygen are small enough to “cluster” around spoilage bacteria, such as Lactobacillus for example, we believe the oxygen nanobubbles migrated to either or both of the bacteria and the proteins in the milk. Because of this migration or complexing with these elements of the milk matrix, the nanobubbles were not free in solution and detectable by the oxygen sensor.

It was unexpected that the oxygen levels in the treated sample would drop below the oxygen levels of the untreated (control) sample. Our initial expectation was that the oxygen level would not drop below the oxygen level in the control group. We believe the oxygen level dropped to the levels in the oxygen control because the oxygen level could not be measured by the oxygen probe once the nanobubbles clustered around either the microbes and/or the proteins in the milk matrix.

The net result on shelf-life of the milk perishable product is significant, in that none of the sniff testers could detect signs of spoilage in the treated sample for an additional ten (10) day period, versus the control. This is an indication that the shelf life of the milk product has been extended by the nanobubble treatment. This occurred despite collective wisdom in the dairy industry that oxygen cannot be used to extend shelf life. The collective wisdom in this industry is that the addition of oxygen will cause negative organoleptic effects and lead to a dairy product that will spoil more quickly.

An advantage of the present oxygen nanobubble treatment embodiments is that the shelf life of the perishable product is extended as a result of the oxygen nanobubbles quickly taking the level of oxygen below the oxygen level of a control solution, as confirmed with an oxygen probe during testing. This is due to a combination of the nanobubbles clustering around the bacteria removing same from the probe's measuring capability. One advantage of this solution to the shelf-life problem is that oxygen is considered GRAS (generally regarded as safe) and accordingly, there should be no requirement to change the label of the milk product as a result of the nanobubble treatment.

It was also identified from our Examples that the oxygen nanobubble treated milk seemed to turn or phase change into a yogurt-like fluid or substance at the end of the trials, instead of becoming sour curds. This is an additional advantage and may be due to nanobubbles interacting with the milk proteins.

Other Examples will be trialed with CO₂ and N₂O nanobubbles in perishable products and liquids.

In view of the foregoing, the present invention includes the following related inventive embodiments (“Emb”), and it is understood that reference to a perishable substance includes reference to a perishable fluid:
Emb 1. A fluid composition having reduced susceptibility to at least one of oxidative spoilage and of microbial spoilage, wherein the fluid composition includes nanobubbles of gas, and each nanobubble in the fluid composition ranges in size from 1 nanometer to 500 nanometers to reduce the at least one of the oxidative spoilage and of the microbial spoilage.
Emb 2. The fluid composition of Emb 1, wherein the gas includes a gas mixture.
Emb 3. The fluid composition of Emb 1, including a food product selected from the group consisting of milk, yogurt, a dairy product other than milk and yogurt, beer, fruit juice, vegetable juice, vegetable oil, vegetable puree, fruit puree, flavored water, and wine.
Emb 4. The fluid composition of Emb 1, wherein the nanobubbles of gas include oxygen nanobubbles.
Emb 5. The fluid composition of Emb 1, wherein the nanobubbles of gas are selected from the group consisting of nitrogen nanobubbles, carbon dioxide nanobubbles, ozone nanobubbles, nitrous oxide nanobubbles, and mixtures thereof.
Emb 6. The fluid composition of Emb 1, including a product selected from the group consisting of latex paint, a cosmetic, liquid soap, and a pharmaceutical.
Emb 7. A process for reducing a fluid composition susceptibility to at least one of oxidative spoilage and of microbial spoilage, including mixing nanobubbles of gas into the fluid composition, and each nanobubble in the fluid composition ranges in size from 1 nanometer to 500 nanometers for reducing the at least one of the oxidative spoilage and of the microbial spoilage.
Emb 8. The process of Emb 7, wherein the gas includes a gas mixture.
Emb 9. The process of Emb 7, wherein the fluid composition includes a food product selected from the group consisting of milk, yogurt, a dairy product other than milk and yogurt, beer, fruit juice, vegetable juice, vegetable oil, vegetable puree, fruit puree, flavored water, and wine.
Emb 10. The process of Emb 7, wherein the nanobubbles of gas include oxygen nanobubbles.
Emb 11. The process of Emb 7, wherein the nanobubbles of gas are selected from the group consisting of nitrogen nanobubbles, carbon dioxide nanobubbles, ozone nanobubbles, nitrous oxide nanobubbles, and mixtures thereof.
Emb 12. The process of Emb 7, wherein the fluid composition includes a product selected from the group consisting of latex paint, a cosmetic, liquid soap, and a pharmaceutical.
Emb 13. The process of Emb 7, further including altering physical characteristics of the fluid composition with the nanobubbles, wherein the physical characteristics are selected from the group consisting of extending the shelf life of the fluid composition, inhibiting bacterial growth in the fluid composition, changing a physical texture of the fluid composition, changing behavior of microbial flora in the fluid composition, and changing characteristics of protein in the fluid composition.
Emb 14. The process of Emb 7, further including removing unwanted gas from the fluid composition before the mixing of the nanobubbles.
Emb 15. The process of Emb 7, further including removing unwanted gas from the fluid composition after the mixing of the nanobubbles.
Emb 16. The process of Emb 7, further including removing unwanted gas from the fluid composition before and after the mixing the nanobubbles.
Emb 17. The process of Emb 13, wherein the changing the physical texture includes changing a viscosity of the fluid composition.
Emb 18. The process of Emb 7, further including scavenging dissolved oxygen and other gases existing in the fluid composition.

Other embodiments (“Emb”) of the present invention may include:
Emb 19. A composition for altering a perishable substance, the composition including nanobubbles present in the perishable substance, wherein the perishable substance is a product selected from the group consisting of milk, yogurt, a dairy product other than milk and yogurt, beer, fruit juice, vegetable juice, vegetable oil, vegetable puree, fruit puree, flavored water, wine, latex paint, a cosmetic, soap, liquid soap, and a liquid pharmaceutical.
Emb 20. The composition of Emb 19, wherein the altering the perishable substance with the nanobubbles is selected from the group consisting of extending the shelf life of the perishable substance, inhibiting bacterial growth in the perishable substance, changing a viscosity and physical texture of the perishable substance, changing behavior of microbial flora in the perishable substance, and changing characteristics of any protein in the perishable substance.
Emb 21. The composition of Emb 19, wherein the nanobubbles are gas selected from the group consisting of oxygen nanobubbles, nitrogen nanobubbles, carbon dioxide nanobubbles, ozone nanobubbles, and nitrous oxide nanobubbles.
Emb 22. A process for altering physical characteristics of a perishable substance, including admixing nanobubbles into the perishable substance, wherein the perishable substance is a product selected from the group consisting of milk, yogurt, a dairy product other than milk and yogurt, beer, fruit juice, vegetable juice, vegetable oil, vegetable puree, fruit puree, flavored water, wine, latex paint, a cosmetic, liquid soap, and a liquid pharmaceutical
Emb 23. The process of Emb 22, wherein the altering the physical characteristics of the perishable substance with the nanobubbles is selected from the group consisting of extending the shelf life of the perishable substance, inhibiting bacterial growth in the perishable substance, changing a physical texture of the perishable substance, changing behavior of microbial flora in the perishable substance, and changing characteristics of any protein in the perishable substance.
Emb 24. The process of Emb 22, wherein the nanobubbles are gas selected from the group consisting of oxygen nanobubbles, nitrogen nanobubbles, carbon dioxide nanobubbles, ozone nanobubbles, and nitrous oxide nanobubbles.
Emb 25. The process of Emb 22, further including at least one of removing unwanted gas from the perishable substance before the admixing the nanobubbles, removing unwanted gas from the perishable substance after the admixing the nanobubbles, and removing unwanted gas from the perishable substance before and after the admixing the nanobubbles.
Emb 26. The process of Emb 23, wherein the changing the physical texture of the perishable substance includes changing a viscosity of the perishable substance.

The present embodiments are directed as well to perishable fluids which include liquids and semi-solid state or phase substances such as for example yogurt which are susceptible to at least one of oxidative spoilage and of microbial spoilage. Examples of such perishable fluids include but are not limited to dairy products, oils, latex paring, cosmetics, liquid soap, and liquid pharmaceuticals.

It will be understood that the embodiments described herein are merely exemplary, and that a person skilled in the art may make variations and modifications without departing from the spirit and scope of the invention. All such variations and modifications are intended to be included within the scope of the invention as provided for herein. It should be understood that the embodiments described above are not only in the alternative but can be combined.

What is claimed is:

1. A fluid composition having reduced susceptibility to at least one of oxidative spoilage and of microbial spoilage, wherein the fluid composition comprises: nanobubbles of gas, and each nanobubble in the fluid composition ranges in size from 1 nanometer to 500 nanometers to reduce the at least one of the oxidative spoilage and of the microbial spoilage.

2. The fluid composition of claim 1, wherein the gas comprises a gas mixture.

3. The fluid composition of claim 1, wherein the fluid composition comprises a food product selected from the group consisting of milk, yogurt, a dairy product other than milk and yogurt, beer, fruit juice, vegetable juice, vegetable oil, vegetable puree, fruit puree, flavored water, and wine.

4. The fluid composition of claim 1, wherein the nanobubbles of gas comprise oxygen nanobubbles.

5. The fluid composition of claim 1, wherein the nanobubbles of gas are selected from the group consisting of nitrogen nanobubbles, carbon dioxide nanobubbles, ozone nanobubbles, nitrous oxide nanobubbles, and mixtures thereof.

6. The fluid composition of claim 1, wherein the fluid composition comprises a product selected from the group consisting of latex paint, a cosmetic, liquid soap, and a pharmaceutical.

7. A process for reducing a fluid composition susceptibility to at least one of oxidative spoilage and of microbial spoilage, comprising: mixing nanobubbles of gas into the fluid composition, and each nanobubble in the fluid composition ranges in size from 1 nanometer to 500 nanometers for reducing the at least one of the oxidative spoilage and of the microbial spoilage.

8. The process of claim 7, wherein the gas comprises a gas mixture.

9. The process of claim 7, wherein the fluid composition comprises a food product selected from the group consisting of milk, yogurt, a dairy product other than milk and yogurt, beer, fruit juice, vegetable juice, vegetable oil, vegetable puree, fruit puree, flavored water, and wine.

10. The process of claim 7, wherein the nanobubbles of gas comprise oxygen nanobubbles.

11. The process of claim 7, wherein the nanobubbles of gas are selected from the group consisting of nitrogen nanobubbles, carbon dioxide nanobubbles, ozone nanobubbles, nitrous oxide nanobubbles, and mixtures thereof.

12. The process of claim 7, wherein the fluid composition comprises a product selected from the group consisting of latex paint, a cosmetic, liquid soap, and a pharmaceutical.

13. The process of claim 7, further comprising altering physical characteristics of the fluid composition, wherein the physical characteristics are selected from the group consisting of extending the shelf life of the fluid composition, inhibiting bacterial growth in the fluid composition, changing a physical texture of the fluid composition, changing behavior of microbial flora in the fluid composition, and changing characteristics of protein in the fluid composition.

14. The process of claim 7, further comprising removing unwanted gas from the fluid composition before the mixing of the nanobubbles.

15. The process of claim 7, further comprising removing unwanted gas from the fluid composition after the mixing of the nanobubbles.

16. The process of claim 7, further comprising removing unwanted gas from the fluid composition before and after the mixing the nanobubbles.

17. The process of claim 13, wherein the changing the physical texture comprises changing a viscosity of the fluid composition.

18. The process of claim 7, further comprising scavenging dissolved oxygen and other gases existing in the fluid composition.
